(19) United States
(12) Patent Application Publication
(10) Pub. No.: US 2010/0305494 A1
(43) Pub. Date: Dec. 2, 2010

(54) METHOD FOR PHOTODYNAMIC THERAPY AND APPARATUS THEREFOR

(76) Inventors: David John Clements, West Sussex (GB); Gavin John Pearson, West Sussex (GB); Jill Ann Williams, West Sussex (GB); Michael John Colles, West Sussex (GB)

Correspondence Address: FAY SHARPE LLP, 1228 Euclid Avenue, 5th Floor, The Halle Building, Cleveland, OH, 44115, US

(21) Appl. No.: 12/530,983

(22) PCT Filed: Mar. 17, 2008
(86) PCT No.: PCT/GB08/50189

§ 371 Date: May 11, 2010

(30) Foreign Application Priority Data
Mar. 15, 2007 (GB) 0704982.8

(51) Int. Cl. A61M 37/00; A61L 2/08

(52) U.S. Cl. 604/20; 422/243; 422/292; 422/22

(57) ABSTRACT

Photodynamic therapy is used in the treatment of orthopaedic infections. Particularly, it is used in the treatment of infections related to orthopaedic conditions and surgery and includes treatment of sites of deterioration or trauma, such as prior to and after prosthetic fixation such as joint replacement or treatment of trauma sites such as pinning of bone fractures. An orthopaedic disinfection apparatus includes a photosensitiser and a light delivery system capable of producing and delivering light at a wavelength which is capable of being absorbed by the photosensitiser. Methods of treatment of orthopaedic infections are also disclosed.

The present invention relates to improvements in or relating to photodynamic therapy, particularly in the treatment of orthopaedic infections. In particular, it relates to the treatment of infections related to orthopaedic conditions and surgery and includes treatment of sites of deterioration or trauma, such as prior to and after prosthetic fixation such as joint replacement or treatment of traumic injuries such as open wounds and including pinning of bone fractures. We will describe a procedure and apparatus suitable for use both in routine pre-treatment situations and, for example, at the time of fixation procedures. The present invention is suitable for use with hard and soft tissue, such as are present in open wounds. As such, it finds particular utility in both civilian and military medical procedures.

Under normal conditions, the immune defence system in the body can control bacterial infections and rapidly responds to any bacterial invasion. Unfortunately, implantable materials such as joint replacements, such as replacement hips, frequently act as sites where bacterial colonies may become established. They do not then succumb to the normal immunological response mechanism so readily. This can lead to serious problems, often affecting the stability of the implant, frequently resulting in eventual surgical revision. This can also lead, if the bacteria become well established, to systemic problems. Similarly, joint capsules may become infected as the blood supply within the capsule is limited and this in turn can result in a focus of infection, which is initially localised but may produce systemic effects with time.

It is estimated that, in 2001, around one million hip and 250,000 knee replacement procedures were carried out worldwide and that around 10% of all hospitalised patients have an indwelling urinary catheter. Although the proportion of procedures which lead to infection is relatively small, the large number of operations carried out mean that the numbers of patients who receive an infection is nevertheless large and the infections themselves are very significant.

In some studies, around 90% of wounds, including traumatic injuries and fixations, which are considered to be clean at the time of closure have contained pathogenic bacteria such as S. aureus. Implant infections are extremely resistant to antibiotics and host defences and implant removal is frequently the only remedy.

Elimination of bacteria from implantable material is particularly difficult once the colonies are established. Current treatment is designed to prevent these colonies developing. It involves the use of prophylactic antibiotic therapy at the time of surgery and for a period afterwards together with impregnation of the cementing medium for the implant with either strings or beads containing antibiotics. In the case of arthroscopy, the capsule is frequently flushed out with isotonic solutions containing soluble sources of antibiotics.

While these methods may be effective, there is a problem that antibiotics are frequently specific to a particular bacterial strain, but the use of broad-spectrum antibiotics to overcome this problem can potentially lead to resistant strains of bacteria being established. In practice therefore, broad-spectrum antibiotics are avoided and the specific antibiotic used may not be matched to the bacterial strains present and so the treatment proves to be ineffective.

There will also be a need for further antibiotic cover should any further surgical intervention be required, in order to prevent re-infection.

The consequences of infection are that the supporting tissues are frequently damaged such that the fixation of the prosthetic device becomes unstable. The consequence of this is that frequently the whole operation has to be repeated. However, the success rate of revisions is not as high as for original operations.

Accordingly, there is a need to provide an alternative system for bacterial killing in orthopaedic uses.

In its broadest sense, the present invention provides an orthopaedic disinfection apparatus comprising a photosensitiser and a light delivery system capable of producing and delivering light at a wavelength which is capable of being absorbed by the photosensitiser.

In a second aspect, the present invention provides a method of disinfecting an orthopaedic site, the method comprising the steps of gaining access to the site, applying a photosensitiser to the site irradiating the photosensitiser with light from a light source at a wavelength which is absorbed by the photosensitiser.

The photosensitiser is preferably selected as having an uptake response appropriate to the target bacteria. Suitably, the photosensitiser is capable of absorbing light towards the red end of the visible spectrum or at longer wavelengths.

Suitably, the photosensitiser is selected from dyes and other photosensitising compounds including azure blue cert, azure B chloride, azure 2, azure A chloride, azure B tetrafluoroborate, thionin, azure A eosinate, azure B eosinate, azure mix sicc, azure II eosinate, arianor steel blue, toluidine blue O, tryptan blue, crystal violet, methylene blue, porphyrins, including haematoporphyrin HCl and haematoporphyrin ester and the porphyrins developed by Destiny Pharma Limited as their XF drugs, phthalocyanines, including aluminium disulphonated phthalocyanine, phenothiazines and chlorines; conjugates, particularly conjugates of these materials, such as nanoparticle tiopronin gold nanoparticulate conjugates, or metabolic precursors of any of these materials. One such metabolic precursor is 5-amino-levulinic acid (ALA) which is metabolised by eukaryotic cells to protoporphyrin IX, a very active endogenous sensitiser.

Preferably, the photosensitising composition comprises at least one photosensitiser selected from toluidine blue O, methylene blue, dimethylene blue or azure blue chloride. More preferably the photosensitiser is toluidine blue O. More preferably, the sensitiser is toluidine blue O in the form of ‘tolonium chloride’, being the pharmaceutical grade of TBO wherein the purity and isometric ratios are maintained.

Advantageously, the photosensitiser is in a form adapted for the treatment site and is adapted to an appropriate method of delivery to maximise contact with the target species in the shortest possible time. Suitably, the photosensitiser is in the form of a liquid, suitably a sprayable liquid, gel or varnish, typically aqueous. A gel may be formed by including a gelling agent in an aqueous solution of the photosensitiser. Suitable gelling agents include hydrophilic polymers such as cellulose derivatives and polyvinyl pyrrolidone. Suitably, the gelling or thickening agent is hydroxypropyl methyl cellulose, typically in an amount of up to about 5% by weight.

The concentration of photosensitiser and the power of light source are selected to provide maximum tissue penetration and kill rates and will be dependent upon the treatment. Suitably, the dye concentration is from 0.00001% to 5%, preferably from 0.00001% to 0.5% w/v, more preferably from 0.001% to 0.1% w/v.

Alternatively, the photosensitiser is supplied as a concentrate for application to the treatment site with dilution in situ to the desired concentration.

Preferably, the light delivery system comprises a light source and a light guide for delivery of light to the photosensitiser.

In one embodiment the light guide is an elongate optical fibre terminating in a tip adapted to illuminate a treatment site in a desired illumination pattern.

In an alternative embodiment, the light delivery system further comprises a handpiece having a distal end to which to which the light guide is demountably attachable.

In a further alternative embodiment, the light guide is formed integrally with a handpiece.

In a first arrangement, the light source is mounted within a housing to which the handpiece is operatively coupled. Suitably, said coupling is by means of an optical fibre. Preferably, the optical fibre is demountably connectable to a proximal end of the handpiece.

In a second arrangement, the light source is mounted within the handpiece.

Suitably, the light source is a laser light source or a light emitting diode light source. The laser light source may be a single laser diode or an array of laser diodes. The light emitting diode light source may be a single light emitting diode, a light emitting diode array or a multiplexed array of light emitting diodes.

Conveniently, the light source is a light emitting diode light source, preferably a single light emitting diode.

Suitably, the light source produces light having a wavelength of from 500 nm to 750 nm, preferably from 600 to 700 nm, more preferably from 615 to 690 nm.

Preferably, the handpiece comprises cooling means adapted to reduce transmission of heat to the light guide. Typically, the handpiece cooling means is adapted to cool the light-emitting diode light-source.

In one embodiment, the handpiece cooling means comprises a heat pipe. Preferably, the handpiece cooling means further comprises a heatsink

Preferably, the optical apparatus further comprises a control console in communication with the handpiece. Suitably, the control console includes heat dissipation means in communication with the handpiece cooling means. Conveniently, the cooling means comprises water as a coolant.

Preferably, the light guide comprises an optical fibre.

In one embodiment, the light guide is of substantially uniform cross-section from a proximal end to a distal end. In an alternative embodiment, the light guide is shaped to taper between the proximal and distal ends. Said tapering may be substantially uniform along the length of the light guide or may be substantially in a distal portion of the light guide alone.

Advantageously, the distal portion of the light guide is shaped to suit the intended purpose of the apparatus. In certain embodiments, the distal portion has a rounded, cylindrical or tapered form. In alternative embodiments, the distal portion has a chisel or wedge form.

Preferably, the apparatus comprises a range of light guides each guide being adapted for a particular use.

Optionally, the light guide further includes a demountable reduction tip to reduce the area of the distal portion or an expansion tip spread light across an increased area or around an increased volume.

In the context of the present invention, the term ‘orthopaedic’ is intended to be interpreted in its broadest sense relating to orthopaedic procedures, including the diagnosis, care and treatment of musculoskeletal disorders, including hard and soft tissue bones, ligaments, muscles and tendons; and skin and connective tissue and medical devices associated with these.

In particular, the present invention provides an apparatus for use against Gram positive and Gram negative bacteria, including strains of Peptostreptococcus, Streptococcus, Staphylococcus, Actinomyces, Bifidbacterium, Coorynebacterium, Eubacterium Lactobacillus, Propioibacterium, Pseudoramibacter, Nieserria, Veillonella, Actinobacilus, Campylobacter, Cantonella, Centipeda, Desulphovibrio, Enterococcus, Escherichia, Fusobacterium, Haemophilus, Porphoromonas, Prevotella, Selenomonas, and Treponema; in particular bacteria selected from Staphylococcus aureus, Staphylococcus epidermidis; Staphylococcus hominis, Staphylococcus haemolyticus, Staphylococcus pyogenes, Pseudomonas aeruginosa, Enterococcus faecalis, Escherichia coli, Propioibacterium acnes, Porphyromonas gingivalis, streptococcus intermedius and Streptococcus mutans; and against spore-forming bacteria such as Bacillus, Clostridium, Sporohalobacter, Anaerobacter and Heliobacterium.

Light may be delivered to the therapy site directly or indirectly by means of the light delivery system. The light delivery system is selected having regard to the nature of the site. For example, the light delivery system may include rigid or flexible light guides and may be adapted to apply light internally to the site or externally as appropriate. In certain circumstances a combination of both internal and external light delivery is appropriate.

In one embodiment, the light delivery system includes a lavage tube adapted to include the light guide. Suitably, the lavage tube includes emitting optical fibres as the light guide. Such an adapted lavage tube can also be used to deliver the photosensitiser during ‘washout’ of the site.

The above and other aspects of the present invention will now be described in further detail, by way of example only, with reference to the accompanying drawings, in which:

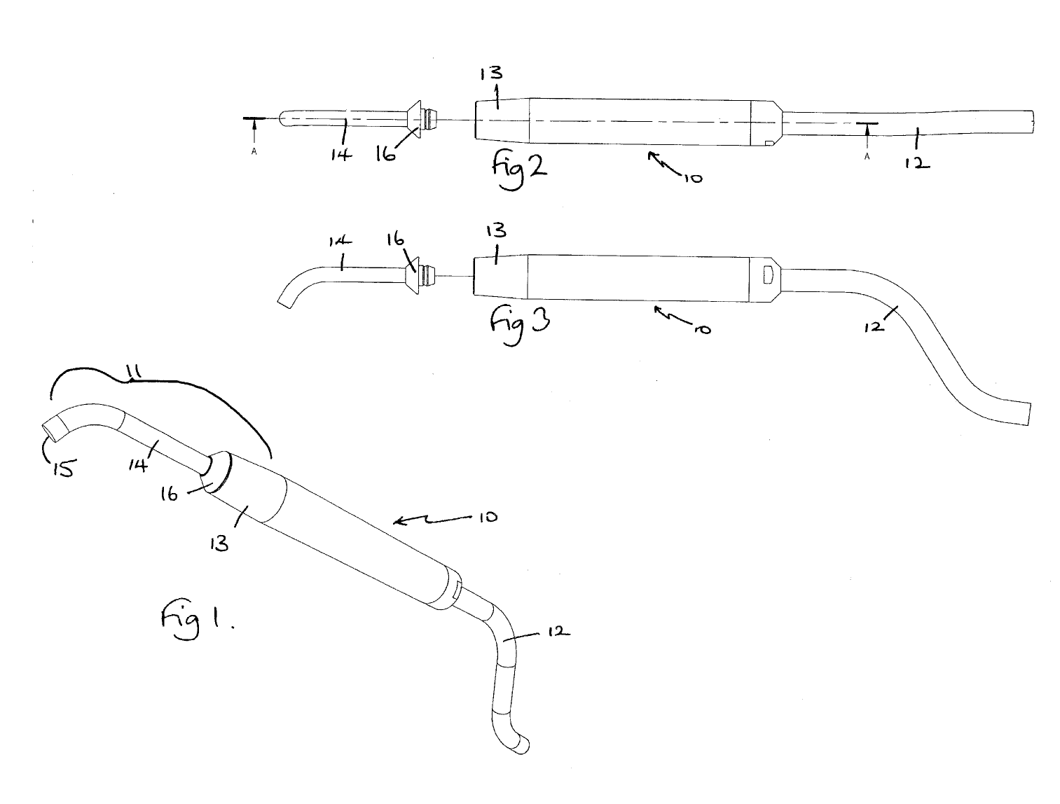

FIG. 1 is a perspective view of a handpiece and light guide of a first embodiment of an orthopaedic disinfection apparatus in accordance with the present invention;

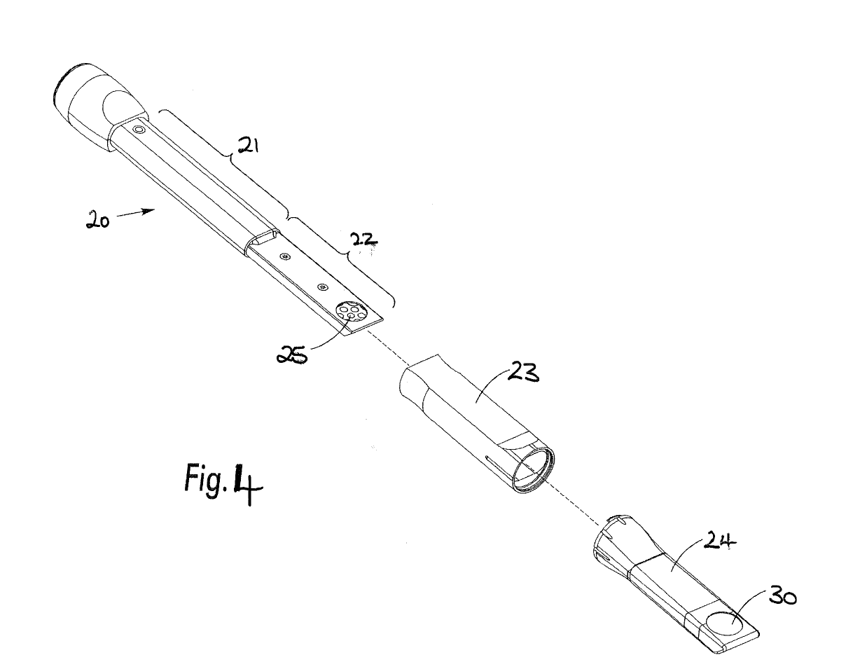

FIG. 2 is plan view of the apparatus to FIG. 1 with light guide element detached;

FIG. 3 is a side view of the apparatus to FIG. 1 with light guide element detached;

FIG. 4 is a perspective disassembled view of a second embodiment of an orthopaedic disinfection apparatus in accordance with the present invention;

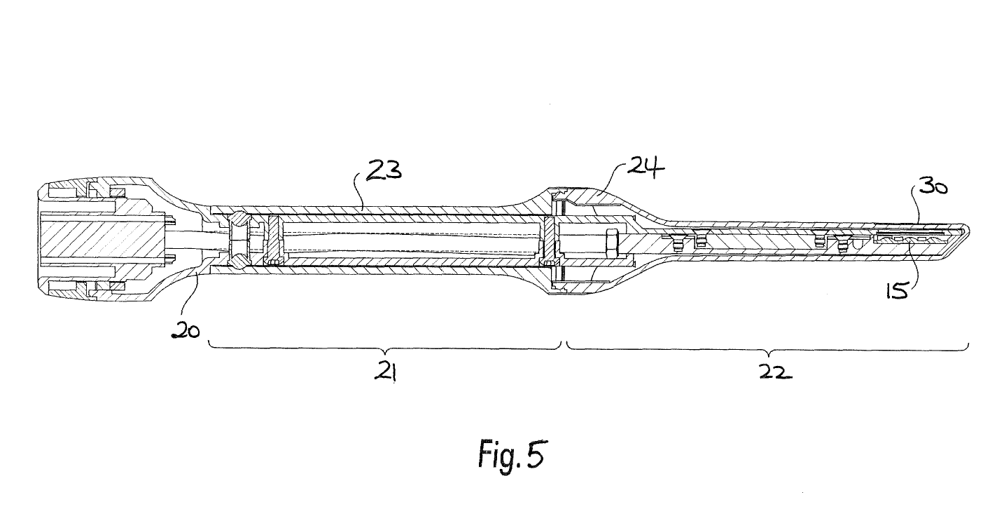

FIG. 5 is a cross-sectional view of the apparatus of FIG. 4 in an assembled configuration.

FIG. 1 shows a handpiece 10 coupled to a light delivery system 11. Handpiece 10 includes an umbilical cord 12 coupled to a control console, not shown. Light delivery system 11 includes a mounting base 13 for operative connection to handpiece 10 and a light guide element 14 for delivery of light to a chosen application site from a distal tip 15 of light guide element 14.

As indicated in FIGS. 2 and 3, the light guide element 14 is demountable from the body of handpiece 10. This enables alternative light guide elements having different lengths and different light focussing or dispersal characteristics to be used, having regard to the desired procedure. The light guide element 14 is mounted at its proximal end within a light guide element clip 16 adapted to be received and retained by a corresponding engagement arrangement at a distal end of handpiece 10 having an external case.

For example, a typical application may use a light guide element 14 having a distal tip 15 with a diameter in the region of 7 mm to 9 mm. Where a greater area is required to be treated, tip diameters of the order of 13 mm may be more advantageous. Where access to the delivery site may be difficult, tip sizes of the order of 3 mm have been found to be successful. Additionally, light guide elements 14 are adapted to provide a range of different light dispersal characteristics. For example, the element can be designed such that light emission is substantially unidirectional or collimated from tip 15 or such that the light is substantially unidirectional but with a degree of off-axis light emission from the tip. Alternatively, the light guide element 14 can be adapted to ‘leak’ light outwardly along a portion, optionally a substantial part of its length, optionally its entire length.

Furthermore, the demountability of the light guide element 14 allows light guide elements to be provided in a range of lengths to suit a range of procedures. For example, light guide elements in the range of 5-7 cm are suitable for intra-oral use, with longer light guide elements being more useful for orthopaedic procedures. Additionally, the light guide element can be changed during a procedure as the light requirements change during the procedure.

The light guide element 14 itself may be manufactured according to any conventional process. Typically, the light guide element is manufactured from a glass optical fibre. The light guide element may be rigid or flexible as required in order to deliver light to the target site. Other optical materials are equally suitably, depending upon the intended use. For example, poly (methylmethacrylate) is also suitable, particularly for substantially straight light guide elements.

The light guide element 14 may also be shaped to suit the requirements of the procedure. In the embodiments shown, the light guide element is curved towards its distal end. Straight elements (not shown) will be equally suitable in certain procedures. The light guide element 14 may be of substantially uniform diameter along its length or may taper towards a narrower diameter at its distal end. In certain embodiments, depending upon application, the light guide element may include a supplementary tip or piece to reduce the distal diameter yet further or a supplementary tip or piece to spread the light across an increased area or volume of the treatment site.

The light guide element can be formed as an elongate element adapted to slide along the treatment site. For example, a light guide element having a long tip can be slid along the side of a bone such as a femur, disinfecting as it is passed along the bone, or along a catheter or cannula. This type of use is particularly suitable for apparatus in which the light source is a laser as it is optically more problematic to avoid attenuation of light in such circumstances with a handpiece-mounted LED arrangement. Elongage element light guide elements need not be provided with a specific handpiece component and can be formed into very long elements, up to full body length or more terminating in a tip having the desired light dispersion properties for the intended therapeutic purpose.

Handpiece 10 may include a light source in the form of a light emitting diode (LED) positioned such that its light output is directed towards light guide element 14. Additionally, handpiece 10 includes electrical supply apparatus (not shown) to provide an electrical supply to the LED. Typically, this includes electrical wires to the umbilical cord 12 and thence to the control console, optionally also including switching apparatus within the handpiece itself to allow ready actuation of the LED by the user.

Alternatively, the light source may be a laser light source, suitably from a laser diode. The light source is suitably housed within the control console and optically coupled to the handpiece by the umbilical cord.

The apparatus preferably also includes passive or active cooling to dissipate heat generated by the LED. Active cooling is suitably achieved by means of a water-cooling system in which coolant is circulated by means of a pump housed within the control console via umbilical cord 12.

With reference to FIGS. 4 and 5, there is shown an alternative embodiment of an orthopaedic disinfection apparatus in accordance with the present invention. The apparatus comprises a handpiece 20 having a proximal portion 21 and a distal portion 22 and a handpiece cover comprising a sleeve 23 for the proximal portion 21 of the handpiece 20 and a sheath 24 for the distal portion of the handpiece 20.

Handpiece 20 includes a light source 25 in the distal portion of the handpiece. In the preferred arrangements, the light source comprises an LED (light-emitting diode) or, more preferably, an array of LEDs. A particularly suitable light source comprises a multiplexed array of LEDs in which multiple LEDs are bundled as a cluster of LEDs and a plurality of clusters are arranged as an array.

Sleeve 23 is adapted to provide a cover for the proximal portion 21 of the handpiece 20, which is the portion which, in use, will be held by the surgeon. The sleeve will, accordingly, typically be made from a hard anodised autoclavable material. However, the sleeve 23 may, alternatively, be manufactured from a disposable material such as a plastics material.

Sheath 24 provides a cover for the distal portion of the handpiece 20, which is the portion from which the light is, in use, emitted. Sheath 24 is designed as a disposable component to eliminate cross-contamination between patients. The sheath may be manufactured from a wholly optically-transparent material (in the sense of being optically transparent at the desired wavelengths) or from a non-transparent material, in which case the sheath 24 is provided with an optically transparent or inert window 30.

In an alternative embodiment (not shown), sleeve 23 and sheath 24 are formed as a unitary component, providing a cover for both the proximal and distal portions of the handpiece. Such a unitary component is most suitably manufactured as a wholly disposable element, including, as described above, an optical window 20 as necessary.

The handpiece 20 is operatively coupled to a base unit (not shown) which provides an electrical supply to the light source at the required voltage and current. Typically, the handpiece is detachable from the control unit. Those skilled in the art will be readily able to devise suitable electrical coupling arrangements.

Additionally, the handpiece 20 houses such cooling devices as may be necessary to maintain either the handpiece generally or the light source at the correct operating temperature. For example, in preferred embodiments, the handpiece 20 includes a water circulating system or circuit and the handpiece 20 includes means for operatively coupling the handpiece to a control unit which includes a water-cooling apparatus.

In alternative embodiments, a heat pipe, provides the necessary cooling.

In this embodiment we have sought to use light emitting diodes (LEDs) because they are available as compact low-cost sources. As described above, they are available as multiplexed arrays, typically comprising 600 or more individual LEDs, with substantial output powers. The output wavelength spread of such devices, whilst not nearly as narrow as that of a laser, are still substantially narrower than any other parameter of significance such as the absorption profile of Toluidine Blue O (TBO) or variations in hard and soft tissue transmission. For example, a particularly suitable LED array is that supplied by Lamina Ceramics under their trade name BL-2000 series. That array provides an output in a broad band which generally coincides with the shoulders of the activation peak of tolonium chloride. The band is less precise than that achievable with a laser diode, but this reduced effectiveness is more than adequately counterbalanced by power outputs of LEDs.

### Experimental Data

An orthopaedic disinfection apparatus of the construction illustrated in FIG. 1 was selected, having a diode laser light source in a control unit, the light source operating at 635 nm and coupled to the handpiece by an umbilical cord. The light was delivered down a light guide fibre to a spherical emitter which was surrounded by a hemispherical reflector of similar diameter to the bacterial plate disc to spread the light across the bacterial plate.

S aureus and S Epidermis were obtained from the Institute of Infection and Immunity, Nottingham. Each strain was subcultured and preserved in 15% glycerol in Tryptone Soya Broth {TSB} as frozen stock at −80° C. For each experiment, colonies of each bacterial species were cultured in TSB and then phosphate buffered saline to produce CFU concentrations in region of 2×10⁷ CFU/ml for S Aureus and 7.6×10⁶ CFU/ml for S epidermis.

50 μl of the bacterial suspension were placed on stainless steel discs (thereby simulating fixation devices) of 15 mm diameter and either 20 μl Tolonium chloride (TBO) [at a concentration of 12.7 μg/ml] or 20 μl Phosphate buffered saline were then spread over the surface using a sterile loop. The solution and bacterial suspension were then left for two minutes. After this, the surface was then irradiated with laser light at 635 nm.

A range of energy outputs and exposure times were evaluated. Following irradiation, the samples were transferred to sterile glass beakers in 10 ml of pre-warmed sterile distilled water. The samples were sonicated for 10 min to remove bacteria adhering to the metal surface. Serial dilutions were carried out and plated onto TSA plates and incubated overnight. Colonies were counted the following day. The results are shown in Table 1.

TABLE 1

| | | | |
|---|---|---|---|
| Laser output | Exposure time | Energy delivered | |
| [mw] | [sec] | [J] | % kill |

S Aureus - TBO

| | | | |
|---|---|---|---|
| 50 | 30 | 1.5 | 41.0 |
| 50 | 90 | 4.5 | 99.45 |
| 50 | 150 | 7.5 | 99.99 |
| 50 | 240 | 12 | 99.99 |
| 50 | 300 | 15 | 99.99 |
| 75 | 20 | 1.5 | 79.60 |
| 75 | 60 | 4.5 | 99.30 |
| 75 | 100 | 7.5 | 99.99 |
| 75 | 160 | 12 | 99.99 |
| 75 | 200 | 15 | 99.99 |
| 100 | 15 | 1.5 | 79.20 |
| 100 | 45 | 4.5 | 99.98 |
| 100 | 75 | 7.5 | 99.99 |
| 100 | 120 | 12 | 99.99 |
| 100 | 150 | 15 | 99.99 |

S epidermis - TBO

| | | | |
|---|---|---|---|
| 50 | 30 | 1.5 | 86.42 |
| 50 | 90 | 4.5 | 99.24 |
| 50 | 150 | 7.5 | 99.97 |
| 75 | 20 | 1.5 | 90.79 |
| 75 | 60 | 4.5 | 99.97 |
| 75 | 100 | 7.5 | 99.97 |
| 100 | 15 | 1.5 | 91.05 |
| 100 | 45 | 4.5 | 99.97 |
| 100 | 75 | 7.5 | 99.97 |

S epidermis Phosphate buffered saline/light (Control)

| | | | |
|---|---|---|---|
| 50 | 150 | 7.5 | 2.63 |
| 100 | 150 | 15 | 25.02 |

Similar results were found when a Perspex disc was substitutes for the stainless steel disc as the substrate for the bacterial suspension.

Other susceptible bacterial include E coli which has been show to be killed under similar conditions. Bacterial suspensions in planktonic solution were prepared and a similar volume of the photosensitiser, tolonium chloride, was added at concentration of 25.4 μg/ml. The solution was gently shaken for 60 seconds and then irradiated with 635 nm laser light for 120 seconds. The bacterial suspension was agitated during the irradiation process. Kill levels in the order of 99.99% have been recorded.

Further tests were carried out using S mutans as the target species with tolonium chloride solution at a final concentration of 13 μg/ml final concentration and a 100 mW output laser diode light source with both spherical and tapered (15 mm long) tip light guides to determine kill levels. The photosensitiser was left in contact with the planktonic solution of S mutans for 60 seconds prior to irradiation. Exposure for the spherical tip was 60 seconds and 150 seconds for the tapered tip. The results are set out in Table 2.

TABLE 2

| | | | | | |
|---|---|---|---|---|---|
| Exposure/ | cfu/ml | cfu/ml | | | log |
| secs | L−S− | L+S+ | % alive | % kill | reduction |

Spherical Tip

| | | | | | |
|---|---|---|---|---|---|
| 60 | 1.09E+008 | 1.00E+002 | 0.0001 | 99.999 | 6.04 |
| 60 | 4.38E+008 | 7.95E+003 | 0.0018 | 99.999 | 4.74 |
| 60 | 2.02E+008 | 7.85E+002 | 0.0004 | 99.999 | 5.41 |
| 60 | 6.30E+007 | 2.20E+003 | 0.0035 | 99.999 | 4.46 |
| 60 | 2.40E+008 | 2.43E+003 | 0.001 | 99.999 | 4.99 |

Mean % kill = 99.999%
log reduction = 5.13
Tapered tip (15 mm long)

| | | | | | |
|---|---|---|---|---|---|
| 150 sec | 2.29E+008 | 0.00E+000 | 0 | 100 | 8.36 |
| 130 sec | 1.80E+008 | 4.00E+001 | 0.00002 | 99.999 | 6.65 |
| 130 sec | 2.62E+008 | 0.00E+000 | 0 | 100 | 8.42 |
| 150 sec | 1.58E+008 | 0.00E+000 | 0 | 100 | 8.2 |
| 150 sec | 2.80E+008 | 0.00E+000 | 0 | 100 | 8.45 |

Mean % kill = 100
log reduction = 8.02

The present is particularly useful in the following orthopaedic fields:
i) prophylactic treatment of the internal surface of the bone and/ or an implant before implant fixation, such as in joint replacement therapy, for example hip-replacements;
ii) treatment of infected orthopaedic periprosthetic infection sites generally, particularly to infected sites prior to revision;
iii) local treatment of joints and joint capsules, using a minimal intervention technique;
iv) arthroscopy, including diagnostic techniques and minimally invasive surgical intervention;
v) septic arthritis, where the current risks of antibiotic resistance is a major issue;
vi) isolated tumours in bone; and
vii) fixations; and
viii) open wounds, including where orthopaedic trauma has occurred.

For example, in this invention, the operating site for placement of an implant such as a hip, knee, elbow, shoulder, finger or toe will be disinfected at the time of surgery. Access to the site will be gained by appropriate methods such as conventional surgery or more minimally-invasive techniques. The photosensitiser is applied to the site using means appropriate to the site and its accessibility and the photosensitiser is then “activated” by application of light from the light source.

Delivery of the light will differ depending on the light source used and the target site. The light may be applied directly from the source or more suitably delivered by a light guide proximally coupled to the light source. The light guide may, for example, be an optical fibre or bundle of optical fibres. At the distal end, the optical fibre is shaped and is dimensioned such that the emitting surface is in close proximity to the area to be treated eg., a cylindrical diffuser matched to the internal dimension of a femur for hip prostheses and a spherical emitter for the treatment of the acetabular cup region in the pelvis. The emitter shape is customised for each application. Alternatively, the light can be delivered directly from a set of light emitting diode arrays, so arranged mechanically as to allow their configuration as a cylinder approximating the dimensions of the femur cavity.

Typical bacterial infections to which the present invention is particularly directed include Staphylococcus aureus and Staphylococcus epidermidis, which together account for around two thirds of implant-related infections; other Staphylococci (about 13% of implant-related infections), Pseudomonas aeruginosa (8%) and Enterococcus faecalis (5%). Escherichia and Streptococcus infections account for a rather smaller number of infections in implants (approximately 2% each).

The inventive photosensitiser/light combination may be applied i) to disinfection of the site at the time of operation; ii) at a subsequent occasion, via minimal access cavity to the site of infection; or iii) to the implants prior to cementation.

The photosensitisers are generally non-toxic to the target microbes in the concentrations envisaged and particularly to the surrounding tissues. However, there is no absolute requirement that the photosensitiser is not toxic to microbes. It is preferred that the photosensitiser used is capable of absorption at the red end of the spectrum or at longer wavelength for greater penetration of the tissues. The preferred photosensitisers are effective against a broad range of both Gram positive and negative bacteria, in particular Staph strains which are frequently associated with bony lesions.

Advantageously, the photosensitiser is in a form adapted for the treatment site and is adapted to an appropriate method of delivery to maximise contact with the target species in the shortest possible time. Suitably, the photosensitiser is in the form of a liquid, suitably a sprayable liquid, gel or varnish, typically aqueous. A gel may be formed by including a gelling agent in an aqueous solution of the photosensitiser. Suitable gelling agents include hydrophilic polymers such as cellulose derivatives and polyvinyl pyrrolidone. Suitably, the gelling or thickening agent is hydroxypropyl methyl cellulose, typically in an amount of up to about 5% by weight.

Of the currently preferred dyes, an aqueous solution of tolonium chloride is the most preferred. Isotonic solutions are preferred, but this is not essential. The concentration of dye range from 0.00001% to 5%. The most preferred concentrations are 0.001%-0.1%.

To provide adequate disinfection will typically require the light source to deliver energy doses to the treatment site between 1 and 500 J/cm³; that is the required energy flux within the volume of infected tissue. The precise dose needed depends on the specific bacteria to be eliminated and the complexity of the application site. For example, photo-activated disinfection with TBO as envisaged in this invention kills Streptococcus mutans with volumetric energy densities in the range 4-40 J/cm³ when the bacteria are in accessible laboratory conditions. Under in-vivo conditions with difficult access or obstructive tissue this figure becomes 100-300 J/cm³.

For example, treatment of an infected knee may preferably be addressed through external application of light from an array of light emitting diodes. Thus relatively low power light applied externally for a relatively long time that may extend to hours can be used to achieve the required volumetric dose within the buried infected site.

Alternatively, such an application under different management conditions may require the fibre-optic delivery of light from a laser source directly to the buried site for shorter times of minutes in order to achieve the required dose.

A further illustrative example is disinfection of a cavity within a femur prepared to accept and prior to the placement of a hip prosthesis. Under the circumstances of this example it is more appropriate to describe the requirements of this invention in terms of the energy doses per unit area of exposed femur surface required to provide satisfactory disinfection. Dependent on the bacteria involved such doses would be within the range 1-400 J/cm². Preferably such doses would be between 5 and 100 J/cm2 and more preferably still between 10 and 50 J/cm².

A yet further example lies in a more generalised use during a procedure in order to ensure disinfection of an area of exposed tissue surrounding and including, but not limited, to the prepared cusp or femur. Under these conditions, light of appropriate wavelength may be projected so as to both cover the site whilst allowing surgery to continue. Additionally, for certain procedures, a lavage tube is adapted to include the light guide, such as emitting optical fibres. Such an adapted lavage tube can also be used to deliver a suitable form of the photosensitiser during ‘washout’ of the site.

There will be a contact time between the photosensitiser and the tissue prior to light application to ensure that the photosensitiser is absorbed onto the bacterial cell wall. Contact times for bacteria with a non-viscous photosensitiser solution at the concentrations cited here are typically between 1 and 300 seconds more typically between 20 and 200 seconds and most typically between 30 and 90 seconds. Where the photosensitiser is applied in a more viscous form, such as a gel, the contact time may be increased as required. Where the photosensitiser is delivered in, for example, an adapted lavage tube, such delivery may be continuous or quasi-continuous and contemporaneous with the application of the light.

1. An orthopaedic disinfection apparatus comprising a photosensitiser and a light delivery system capable of producing and delivering light at a wavelength which is capable of being absorbed by the photosensitiser.

2. An orthopaedic disinfection apparatus as claimed in claim 1 wherein the photosensitiser is selected as having an uptake response appropriate to the target bacteria.

3. An apparatus as claimed in claim 1 wherein the photosensitiser is selected from dyes and other photosensitising compounds including azure blue cert, azure B chloride, azure 2, azure A chloride, azure B tetrafluoroborate, thionin, azure A eosinate, azure B eosinate, azure mix sicc, azure II eosinate, arianor steel blue, toluidine blue O, tryptan blue, crystal violet, methylene blue, porphyrins, including haematoporphyrin HCl and haematoporphyrin ester, phthalocyanines, including aluminium disulphonated phthalocyanine, phenothiazines and chlorines; conjugates, particularly conjugates of these materials, such as nanoparticle tiopronin gold nanoparticulate conjugates, or metabolic precursors of any of these materials.

4. An apparatus as claimed in any claim 1 wherein the photosensitiser is in a form adapted for the treatment site and is adapted to an appropriate method of delivery to maximise contact with the target species in the shortest possible time.

5. An apparatus as claimed in claim 4 wherein the photosensitiser is in the form of a liquid, suitably a sprayable liquid, gel or varnish, preferably aqueous.

6. An apparatus as claimed in claim 1 wherein the light delivery system comprises a light source and a light guide for delivery of light to the photosensitiser.

7. An apparatus as claimed in claim 6 wherein the light delivery system further comprises a handpiece having a distal end to which to which the light guide is demountably attachable.

8. An apparatus as claimed in claim 6 wherein the light guide is formed integrally with a handpiece.

9. An apparatus as claimed in claim 8 wherein the light source is mounted within a housing to which the handpiece is operatively coupled.

10. An apparatus as claimed in claim 9 wherein said coupling is by means of an optical fibre.

11. An apparatus as claimed in claim 10 wherein the optical fibre is demountably connectable to a proximal end of the handpiece.

12. An apparatus as claimed in claim 9 wherein the light source is a laser light source or a light emitting diode light source.

13. An apparatus as claimed in claim 1 wherein the light source is mounted within the handpiece.

14. An apparatus as claimed in claim 13 wherein the light source is a light emitting diode light source.

15. An apparatus as claimed in claim 12 wherein the light emitting diode light source is a single light emitting diode, a light emitting diode array or a multiplexed array of light emitting diodes, preferably a single light emitting diode.

16. An apparatus as claimed in claim 1 wherein the light source produces light having a wavelength of from 500 nm to 750 nm, preferably from 600 to 700 nm, more preferably from 615 to 690 nm.

17. A method of disinfecting an orthopaedic site, the method comprising the steps of gaining access to the site, applying a photosensitiser to the site irradiating the photosensitiser with light from a light source at a wavelength which is absorbed by the photosensitiser.

18. A method as claimed in claim 17 wherein the photosensitiser and light source are provided by an apparatus as claimed in claim 1.

19. A method as claimed in claim 17 or claim 18 wherein the disinfection treatment is selected from: i) prophylactic treatment of the internal surface of the bone and/or an implant before implant fixation, such as in joint replacement therapy, for example hip-replacements; ii) treatment of infected orthopaedic periprosthetic infection sites generally, particularly to infected sites prior to revision; iii) local treatment of joints and joint capsules, using a minimal intervention technique; iv) arthroscopy, including diagnostic techniques and minimally invasive surgical intervention; v) septic arthritis, where the current risks of antibiotic resistance is a major issue; vi) isolated tumours in bone; and vii) fixations; and viii) open wounds, including where orthopaedic trauma has occurred.
